(19) United States
(12) Patent Application Publication
(10) Pub. No.: US 2022/0031449 A9
(43) Pub. Date: Feb. 3, 2022

(54) CUSTOMIZED OPTICAL LENS BASED ON PATIENT-SPECIFIC MEASUREMENT DATA

(71) Applicant: WAVEPRINT TECHNOLOGIES, Eugene, OR (US)

(72) Inventor: Balamurali K. Ambati, Eugene, OR (US)

(21) Appl. No.: 16/528,486

(22) Filed: Jul. 31, 2019

Related U.S. Application Data
(63) Continuation of application No. PCT/US2018/016312, filed on Jan. 31, 2018.
(60) Provisional application No. 62/452,858, filed on Jan. 31, 2017.

(51) Int. Cl. A61F 2/16

(52) U.S. Cl. CPC A61F 2/1637; A61F 2250/0097; A61F 2240/002

(57) ABSTRACT

Methods for a patient surgically receiving a customized IOL for a particular eye according to patient-specific measurement data are provided. The methods may include preoperative evaluation of a particular eye of a particular patient and accumulation of patient-specific measurement data by a physician and/or a hospital. The physician, designee, and/or hospital may transmit the measurement data for the patient to the customized IOL manufacturer. The IOL manufacturer may manufacture and customize the IOL. The manufacturer may deliver the customized IOL back to the surgeon, designee, and/or hospital, after which the surgeon may perform the surgery.

### CROSS-REFERENCE TO RELATED APPLICATIONS

The present application is a continuation of International Patent Application No. PCT/US2018/016312, filed Jan. 31, 2018, which claims priority pursuant to U.S.C. § 119(e) to U.S. Provisional Patent Application No. 62/452,858, filed Jan. 31, 2017, each of which are hereby incorporated by reference for all purposes.

### TECHNICAL FIELD

Embodiments of the present invention generally relate to optical lenses, and more specifically to an intraocular lens (“IOL”) that may be customized based on patient-specific measurement data.

### BACKGROUND OF THE INVENTION

IOLs are commonly implanted in the eye to treat certain conditions, such as cataracts or myopia. For example, an IOL is implanted in the eye as a replacement for the natural crystalline lens after cataract surgery or to alter the optical properties of an eye in which the natural lens remains. The IOL provides the light focusing function originally undertaken by the crystalline lens. Insertion of an IOL for the treatment of cataracts is the most commonly performed eye surgical procedure. Each year approximately 1.4 million people in the United States alone undergo cataract surgery.

A typical IOL includes an optic or lens body for focusing light toward the retina of the eye. In addition, the IOL also includes one or more fixation members or haptics for securing the IOL in the desired position within the chamber of the eye. The IOL is implanted directly into the eye through a small incision formed in the ocular tissue of the eye. To fit through this small incision, modern IOLs are designed to be deformed, e.g., rolled, folded or the like, to a relatively small profile to pass through the small incision and then allowed to return to their original shape within the eye.

Currently, the IOL selection for patients is limited. With the exception of a few hundred patients who receive lenses that can be adjusted after implantation, the selection requires that the patients choose an IOL with preset spherical and astigmatic power from a library of IOLs, all with preset powers and fixed power increments such as 0.5 D for spherical and 0.75 D for cylindrical. Furthermore, while some IOLs have positive or negative spherical aberration, the aberrations are generally fixed for particular IOLs and hence, cannot be optimized by patient as per their requirements. This limited availability of power limits the IOLs' precision in correcting sphero-cylindrical error and solving the patients' eye care needs.

Accordingly, it is advantageous to have customizable IOLs that exhibit the exact power required by patients.

### SUMMARY OF THE INVENTION

Provided herein are embodiments of IOLs that may be customized according to patient-specific measurement data.

The term “lens” as used herein refers to a transmissive optical device that focuses or disperses a light beam by means of refraction.

The terms “lens blank” as used herein refers to a piece of glass of suitable size, design, and composition for use, when ground and polished, as a lens.

The term “non-transitory medium” as used herein refers to many forms of computer-readable media that store data only for short and/or long periods of time and/or only in the presence of power, such as register memory, processor cache, and Random Access Memory.

The term “customizing” as used herein refers to the process of manufacturing an IOL based on a data set for a particular eye of a particular patient. For example, the data set could include axial length measurement, keratometry, anterior and posterior corneal topography, ocular biometry (white to white, anterior chamber depth), estimated lens position (based on ultrasound biomicroscopy (“UBM”) and/or optical coherence tomography (“OCT”) interferometry), and corneal/higher-order aberrations, visual axis positioning, and/or horizontal meridian registration (using iris or sclera markers).

“UBM” is a technique primarily used for imaging of the anterior segment of the eye. Various structures of the eye can be visualized with UBM, such as the cornea, iris, anterior chamber angle, scleral spur, ciliary body, posterior chamber, anterior chamber, and lens.

“OCT” is a non-invasive imaging test. OCT uses light waves to take cross-section pictures of your retina.

The term “IOL” refers to medical devices that are implanted inside the eye to replace the eye's natural lens when it is removed during an eye surgery, such as cataract surgery.

The “visual axis” is defined as a line passing from the fovea (a small depression in the retina of the eye where visual acuity is highest) through the nodal point of the eye. If the pupil of the eye is displaced from the eye's optical axis, then the visual axis and line of sight may be different.

“Optical biometry” is the current standard for IOL power calculations in clinical practice. Optical biometry is a highly accurate non-invasive automated method for measuring the anatomical characteristics of the eye.

Generally, the observer's eye is considered to be at the center of an imaginary sphere. More precisely, the center of the sphere is in the center of the pupil of the observer's eye. The observer is looking at a point, the “fixation point,” on the interior of the sphere. The “horizontal meridian” runs from an observer's left, through the fixation point, and to the observer's right. The “vertical meridian” runs from above the observer's line of sight (a straight line along which an observer has unobstructed vision), through the fixation point, and to below the observer's line of sight.

The term “customized IOL” refers to an IOL that has a refraction selected based on a data set for a particular eye of a particular patient.

The term “data set” unless otherwise specified refers to a set of measurements of a particular eye of a particular patient.

The term “finalizing” as used herein refers to any combination of the following three processes: a) one or more steps that refine the uniformity of the interior and/or surface of the lens; b) one or more steps that render the lens safe for use in the eye, such as covalently incorporating any remaining diffusive species; and/or c) one or more steps that make the refraction permanent, such as making the material insensitive to further exposure to light.

The term “diffusive” as used herein refers to a chemical species that capable of diffusing in the lens matrix at an appropriate temperature.

The term “tolerance” as used herein, unless otherwise specified, refers to the maximum deviation permitted from a specified correction. For example, if the defocus of the eye must be corrected to within ±0.25D, the tolerance is 0.25D. For another example, if the desired correction includes only defocus and astigmatism, a tolerance may be specified for each of them individually, such as a tolerance of 0.15D for defocus and a tolerance of 0.3D for astigmatism.

The term “matrix” as used herein refers to an optically transparent material that is foldable in the sense of a foldable IOL.

The term “extract” as used herein refers to removing a diffusive chemical species from a lens blank matrix by immersing the lens blank matrix in a solvent that dissolves the chemical species. Subsequently, the lens blank matrix is immersed for a suitable time at a suitable temperature for the diffusive chemical species to diffuse out of the object. The solvent could be a liquid, a supercritical fluid, a gas, or a vacuum. The use of a gas or vacuum environment is appropriate for a diffusive chemical species that is volatile at the suitable temperature. If some of the solvent swell the object, then an appropriate step is included for removing the solvent from the object, such that the diffusive chemical species has been removed and no solvent has been added to the object.

The term “phakic-IOL implantation” as used herein refers to a surgical procedure to correct refractive error by implanting an IOL while leaving the crystalline lens in place. It is an alternative to LASIK or PRK laser eye surgery for patients with presbyopia and high hyperopia (farsightedness). Leaving the crystalline lens in place is advantageous for patients who have accommodative function, where the crystalline lens dynamically adjusts itself to focus between far and near objects. Accommodative function decreases with age until it is negligible. The age at which accommodative function is lost is typically 45 years of age. Phakic-IOL implantation is relevant to patients between 18 and 45 years of age.

The term “presbyopia” refers to the condition in which the crystalline lens is not able to dynamically adjust focus between distance and near.

The term “refractive lens exchange,” sometimes called lens replacement surgery or clear lens extraction, refers to a surgical procedure to correct refractive error by removing the crystalline lens and implanting an IOL. It is an alternative to LASIK, PRK laser eye surgery, or phakic IOL refractive surgery for patients with presbyopia and high hyperopia.

The term “cataract surgery” refers to a surgical procedure to restore vision that is impaired due to cloudiness in the crystalline lens (the transparent elastic structure behind the iris by which light is focused onto the retina of the eye.) by removing the crystalline lens and implanting an IOL.

The term “capsulotomy” as used herein refers to a surgical procedure during cataract surgery (“the removal of the natural lens of the eye that has developed an opacification, which is referred to as a cataract”) that produces a circular incision through the anterior portion of the capsule of the crystalline lens of the eye, leaving the rest of the capsule of the crystalline lens intact.

The term “anterior capsulorhexis” refers to the circular incision through the anterior portion of the capsule of the crystalline lens.

The term “phacoemulsification” as used herein refers to a technique of cataract extraction through the anterior capsulorhexis using high-frequency ultrasonic vibrations to fragment the lens combined with controlled irrigation to maintain normal pressure in the anterior chamber, and suction to remove lens fragments and irrigating fluid. Phacoemulsification allows the crystalline lens to be removed through a small incision.

The term “capsular bag” as used herein refers to the sack-like structure remaining within the eye after the crystalline lens has been removed by phacoemulsification, which also called extracapsular cataract extraction.

The term “capsular polishing” as used herein refers to a surgical procedure during cataract surgery that thoroughly cleans the inner surface of the capsular bag after removal of the crystalline lens.

The term “intraoperative aberrometry” refers to an additional tool that allows surgeons to take both aphakic and pseudophakic refractive measurements in the operating room to aid in the determination of IOL power selection and placement.

The term “higher-order aberrations” is a distortion acquired by a wavefront of light when it passes through an eye with irregularities of its refractive components (tear film, cornea, aqueous humor, crystalline lens and vitreous humor). It may refer to optical aberrations of the eye that are quantified by Zernike polynomials, Hartman-Shack imaging, Fourier transform, or ray tracing methods. 1) Coma is a 3rd order aberration, which can be any linear, horizontal, or vertical in orientation, and clinically produce streaking from a point source of light. 2) Spherical aberration is a 4th order aberration that clinically produces halos around point sources of light. And 3) Trefoil and quadrafoil are 3rd and 4th order aberrations, respectively, which clinically produce starburstings around point sources of light. Some common wavefront aberrations include piston, vertical prism, horizontal prism, astigmatism, defocus, astigmatism, trefoil, vertical coma, horizontal coma, quadrafoil, secondary astigmatism, spherical aberration, and secondary astigmatism. The Zernike polynomials may be characterized using the Optical Society (“OSA”) system or the American National Standards Institute (“ANSI”) system, such as ANSI standard Zernike mode pyramid as illustrated in FIG. 8.

According to some embodiments, an additive process for customizing IOLs is provided. The additive process may include selecting a lens blank; adding material to the anterior surface, the posterior surface, or both surfaces of the lens blank to generate or move refracting surfaces and create a lens; matching the lens with the specifications of a particular eye of a particular patient such as corneal topography, visual axis, corneal aberrations, ocular biometry (white to white, anterior chamber depth), estimated lens position (based on UBM and/or OCT interferometry), and posterior corneal shape with sufficient precision to correct aberrations to a desired order; and finalizing the lens when it matches the specifications of the particular eye of the particular patient within a desired tolerance.

According to some embodiments, a subtractive process for customizing IOLs is provided. The subtractive process may include selecting a lens blank; removing material from the anterior surface, the posterior surface, or both surfaces of the lens blank to generate refracting surfaces and create a lens; matching the lens with a patient's specifications such as corneal topography, visual axis, corneal aberrations, ocular biometry (white to white, anterior chamber depth), estimated lens position (based on UBM and/or OCT interferometry), and posterior corneal shape with sufficient precision to correct aberrations to the desired order; and finalizing the lens when it matches the patient's specifications for the particular eye that will receive the customized IOL within the desired tolerance.

According to some embodiments, an internal additive process for customizing IOLs is provided. The internal additive process may include selecting a lens blank matrix; pairing the lens blank matrix with a diffusive species; impregnating the diffusive species into the lens blank matrix; conducting a spatially-resolved reaction between the diffusive species and the lens blank matrix; allowing the remaining diffusive species to redistribute in the lens matrix; matching the lens with a patient's specifications such as corneal topography, visual axis, corneal aberrations, ocular biometry (white to white, anterior chamber depth), estimated lens position (based on UBM and/or OCT interferometry), and posterior corneal shape with sufficient precision to correct aberrations to the desired order; and finalizing the lens when it matches the patient's specifications for the particular eye that will receive the customized IOL within the desired tolerance.

According to some embodiments, an internal subtractive process for customizing IOLs is provided. The internal subtractive process may include selecting a lens blank matrix; conducting a spatially-resolved reaction to cause some of the matrix to become one or more diffusive species; extracting the diffusive species from the remaining lens blank matrix to modify its shape and/or refractive index; matching the lens with a patient's specifications such as corneal topography, visual axis, corneal aberrations, ocular biometry (white to white, anterior chamber depth), estimated lens position (based on UBM and/or OCT interferometry), and posterior corneal shape with sufficient precision to correct aberrations to the desired order; and finalizing the lens when it matches the patient's specifications for the particular eye that will receive the customized IOL within the desired tolerance.

According to some embodiments, an internal redistributive process for customizing IOLs is provided. The internal redistributive process may include selecting a lens blank matrix, which may include a fully self-contained elastomer with diffusive species; conducting a spatially-resolved reaction to permanently incorporate some of the diffusive species in the lens blank matrix material; allowing the remaining diffusive species to redistribute in response to a composition gradient that was created by the reaction; matching the lens with a patient's specifications such as corneal topography, visual axis, corneal aberrations, ocular biometry (white to white, anterior chamber depth), estimated lens position (based on UBM and/or OCT interferometry), and posterior corneal shape with sufficient precision to correct aberrations to the desired order; and finalizing the lens when it matches the patient's specifications for the particular eye that will receive the customized IOL within the desired tolerance.

According to some embodiments, methods for a patient surgically receiving a customized IOL for a particular eye according to patient-specific measurement data are provided. The methods may include preoperative evaluation of a particular eye of a particular patient and accumulation of patient-specific measurement data by a physician and/or a hospital. The physician, designee, and/or hospital may transmit the measurement data for the patient to the customized IOL manufacturer. The IOL manufacturer may manufacture and customize the IOL. The manufacturer may deliver the customized IOL back to the surgeon, designee, and/or hospital, after which the surgeon may perform the surgery.

This summary and the following detailed description are merely exemplary, illustrative, and explanatory, and are not intended to limit, but to provide further explanation of the invention as claimed. Additional features and advantages of the invention will be set forth in the descriptions that follow, and in part will be apparent from the description, or may be learned by practice of the invention. The objectives and other advantages of the invention will be realized and attained by the structure particularly pointed out in the written description, claims, and the appended drawings.

### BRIEF DESCRIPTION OF THE DRAWINGS

The present invention may be better understood by referring to the following figures. The components in the figures are not necessarily to scale. Emphasis instead should be placed upon illustrating the principles of the disclosure. In the figures, reference numerals designate corresponding parts throughout the different views.

FIG. 1 illustrates an additive process for customizing IOLs according to some embodiments of the present invention.

FIG. 2A illustrates a subtractive process for customizing IOLs according to some embodiments of the present invention.

FIGS. 2B-2E illustrate different lens blanks after undergoing the subtractive process according to some embodiments of the present invention.

FIG. 3 illustrates a block diagram of an internal additive process for customizing IOLs according to some embodiments of the present invention.

FIG. 4 illustrates a block diagram of an internal subtractive process for customizing IOLs according to some embodiments of the present invention.

FIG. 5 illustrates a block diagram of an internal redistributive process for customizing IOLs according to some embodiments of the present invention.

FIG. 6A illustrates a perspective and side view of a customized IOL with positioning holes.

FIG. 6B illustrates a perspective view of a customized IOL with positioning markings.

FIG. 6C illustrates a perspective view of a customized IOL with both positioning holes and positioning markings.

FIGS. 6D-6E illustrate a top view of a customized IOL with positioning holes.

FIG. 7 illustrates a block diagram of the process involved in a patient surgically receiving a customized IOL according to some embodiments of the present invention.

FIG. 8 illustrates an exemplary ANSI standard Zernike mode pyramid.

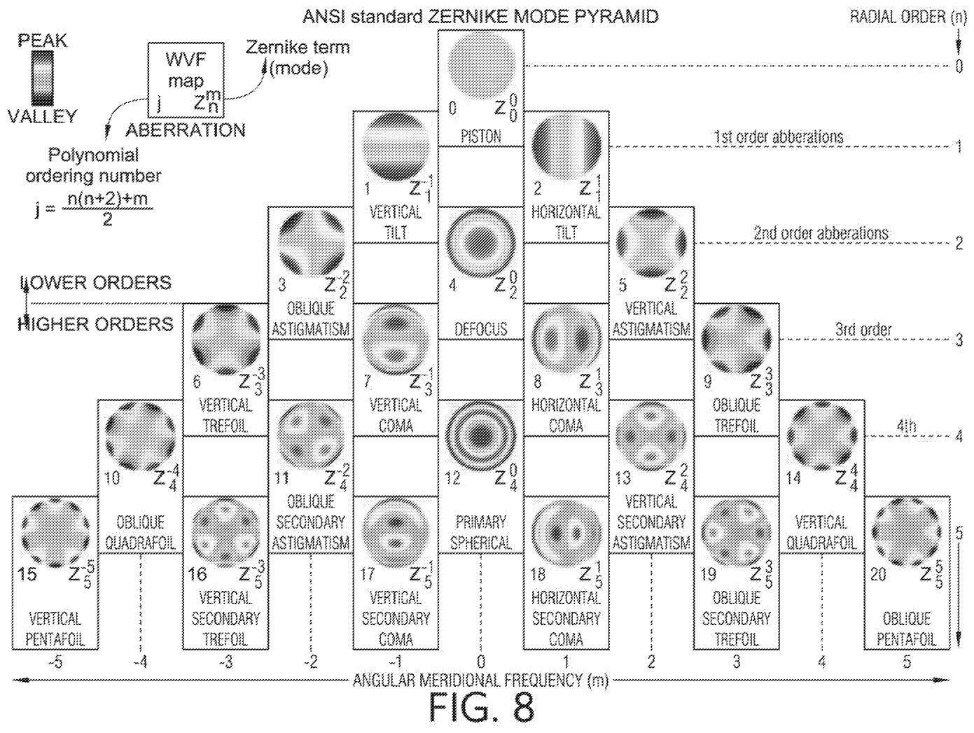

### DETAILED DESCRIPTION

The detailed description below illustrates the described invention and method of use in at least one of its preferred, best mode embodiment, which is further defined in detail in the following description. In particular, the following examples illustrate exemplary customized IOLs and related compositions, methods, systems, and devices. A person skilled in the art will appreciate the applicability and the necessary modifications to adapt the features described in detail in the present section, to additional IOLs, compositions, devices, methods, and systems according to embodiments of the present disclosure. While this invention is susceptible to different embodiments in different forms, a preferred embodiment of the invention will be shown in the drawings and herein described in detail with the understanding that the present disclosure is to be considered as an exemplification of the principles of the invention and is not intended to limit the broad aspect of the invention to the embodiment illustrated. All features, elements, components, functions, and steps described with respect to any embodiment provided herein are intended to be freely combinable and substitutable with those from any other embodiment unless otherwise stated. Therefore, it should be understood that what is illustrated is set forth only for the purposes of example and should not be taken as a limitation on the scope of the present invention.

This invention is enabled by the deployment of devices (e.g., femtosecond laser, rhexis guides (instruments to facilitate a precise capsulorhexis, e.g., verus ring, mynosys zepto, femtplaser, or capsulaser)) that enable reproducible capsulotomy creation with a precision of +/−0.1 mm, aberrometry assessment of cornea separated from internal optics of the eye, centration of intraocular lens to within 0.1 mm of the pupillary center, assessment of posterior corneal cylinder to within 0.1 D, and intraoperative aberrometry to confirm neutralization of refractive error to less than 0.1 D.

Recent advancements in the devices, methods, and systems concerning IOLs and IOL insertion devices have created a need for this invention. Eye surgeons across the world now have the ability to insert IOLs into the eye using an IOL insertion device, such as the one described in U.S. Pat. No. 6,334,862, which is incorporated by reference in its entirety. IOLs are also being manufactured using materials that facilitate their deformation such that they can pass through a small opening. More information on foldable IOLs is provided in U.S. Pat. No. 5,171,319, which is incorporated by reference in its entirety. The availability of flexible and durable materials to manufacture IOLs allows the incision in the eye, made prior to inserting the IOL into the eye, to be small. Unlike the current invention, existing technology, for example as described in U.S. Patent Pub. No. 2005/0099597 relate to IOLs that can be modified post-manufacture using light sources. Specifically, such IOLs are self-contained and do not require the addition or removal of materials to change the optical properties. Instead, the optical properties are altered by exposing a portion or portions of the optical element to an external stimulus that induces polymerization of a modifying composition (“MC”) within the element. The polymerization of the MC, in turn, causes the change in optical properties. Such a post-operative IOL modification requires a costly light delivery device and multiple visits to the surgeon. Moreover, post-operative IOL modification can involve harmful modification sources such as UV rays that can affect the patient's eye and/or health. The present invention overcomes these failings of the prior art. Additionally, advancements in the devices, methods, and systems of eye surgery provide control of the small size and specific position of the incision that needs to be made in order to insert the IOL into the eye effectively. Examples of such devices, methods, and systems are described in U.S. Pat. No. 5,370,652, which is incorporated herein by reference in its entirety. Recently, femtosecond laser systems have emerged as an alternative to manual incisions in the cornea and crystalline lens for different ophthalmic surgeries. Examples of such laser systems are the Intralase FS Laser, IFS Advanced Femtosecond Laser, LenSx Femtosecond Laser, AMO-Catalys, Lensar, and Zeimer Z6 or Z8. Such lasers make incisions by focusing ultrashort laser pulses to a very fine focus, causing a plasma mediated photodisruption of the tissue at the points of focus. The incision is generated by placing a contiguous series of such pulses in the pattern of the desired incision. The combined effect of the pattern of pulses is cleaving the tissue at the targeted plane. Arbitrarily complex incision patterns can be generated with such lasers. Furthermore, femtosecond lasers are believed to make more accurate and consistent incisions than the incisions formed manually. Other devices, methods, and systems to generate incisions in the eye are described in U.S. Patent Publ. No. 2012/0271286, which is incorporated herein by reference in its entirety. In an embodiment, the incisions made by the devices, methods, and systems have a diameter ranging between 1.8 mm to 3.2 mm.

Furthermore, the small size and specific position of the incision ensure that changes in a patient's vision due to healing from the surgery are both limited and predictable. Surgeon induced astigmatism with modern incisions is generally less than 0.2 D with much less variability, whereas 10 or 15 years ago, astigmatism induction was frequently 1 D or more. Limited, predictable changes in the shape of the eye due to the small incision provide much better correlation between post-operative refraction and preoperative measurement of a patient's specific axial length, higher-order corneal aberrations, anterior and posterior corneal topography, visual axis, corneal aberrations, ocular biometry (white to white, anterior chamber depth), estimated lens position (based on UBM and/or OCT interferometry), and astigmatism, and estimated lens position. Finally, now, cataract surgeons have the ability to position IOLs precisely, by controlling the IOLs' centration to within 100 microns, azimuthal orientation to within 3 degrees axial position, and tilt less than 3 degrees. The surgeons position the IOLs precisely using precise creation of the anterior capsulorhexis, such that it is centered on the visual axis, using femtosecond laser, visual axis registration, and/or rhexis guides. They also have the ability to provide a stable site for the IOL's optic using meticulous, interoperative posterior and anterior capsular polishing and minimizing the postoperative capsular fibrosis. The postoperative capsular fibrosis is minimized using intraoperative visualization of IOLs' centration and position using heads-up display systems, for example, Zeiss Callisto, and intraoperative OCT. Intraoperative wavefront aberrometry, such as ORA or Holos, which have come to market within the last 5 years, is used to precisely adjust the centration, rotation, azimuthal orientation, tilt, and axial position to minimize wavefront aberrations before closing the incision. More information on the insertion and placements of IOLs is provided in U.S. Pat. No. 7,878,655, which is incorporated by reference in its entirety. Some IOLS that are designed for post-operative re-positioning and methods for post-operative IOL re-positioning are described in U.S. Pat. No. 5,571,177, which is incorporated by reference in its entirety. Therefore, new IOLs are needed that provide the improved vision that is possible given the new levels of precision in cataract surgery.

This invention addresses the unmet need for IOLs that correct spherical error, astigmatism and higher order aberrations in a patient-customized fashion using preoperative patient-specific measurement data without the risk, complexity, and cost of in situ adjustment. This would be expected to improve contrast sensitivity (measured by modulation transfer function or other contrast sensitivity measures), spatial resolution, and visual acuity and reduce night glare (measured by glaremeter or ocular scatter index) and dysphotopsias. Dysphotopsias are mostly measured by questionnaire, but more recently it has been measured by iTrace or Visiometrics or OPD-3 that do glare simulations using ocular scatter index. Starting in 2004, the LASIK literature started showing improved outcomes, including with respect to contrast sensitivity with corrections of higher order aberrations (“HOAs”). For example, a research evaluated the visual performance of two customized ablation systems (wavefront-guided ablation and topography-guided ablation) in LASIK. In wavefront-guided laser ablation, information obtained from a wavefront-sensing aberrometer (which quantifies the aberrations) is transferred electronically to the treatment laser to program the ablation. The hallmark of topography-guided ablation is that it's designed to address corneal issues exclusively, with an emphasis on smoothing or normalizing the anterior corneal surface. As a result, it's often used to treat corneal abnormalities such as scars or keratoconus. But because it relies on topography for guidance, it arguably accomplishes some things—such as centering the treatment on the line of vision—better than pupil-oriented measuring technologies such as wavefront, even in normal eyes. In addition, it sometimes uses ablation schemes that are significantly different than a wavefront-guided ablation would use to achieve a given result, with potentially significant consequences for the cornea and the eye. In this prospective, randomized clinical study, 68 eyes of 35 patients undergoing LASIK were enrolled. Patients were randomly assigned to wavefront-guided ablation using the iDesign aberrometer and STAR S4 IR Excimer Laser system (wavefront-guided group; 32 eyes of 16 patients; age: 29.0±7.3 years) or topography-guided ablation using the OPD-Scan aberrometer and EC-5000 CXII excimer laser system (topography-guided group; 36 eyes of 19 patients; age: 36.1±9.6 years). Preoperative manifest refraction was −4.92±1.95 diopters (D) in the wavefront-guided group and −4.44±1.98 D in the topography-guided group. Visual function and subjective symptoms were compared between groups before and 1 and 3 months after LASIK. Of seven subjective symptoms evaluated, four were significantly milder in the wavefront-guided group at 3 months. Contrast sensitivity with glare off at low spatial frequencies (6.3° and 4°) was significantly higher in the wavefront-guided group. Uncorrected and corrected distance visual acuity, manifest refraction, and higher order aberrations measured by OPD-Scan and iDesign were not significantly different between the two groups at 1 and 3 months after LASIK. Accordingly, it was concluded that both customized ablation systems used in LASIK achieved excellent results in predictability and visual function. The wavefront-guided ablation system may have some advantages in the quality of vision. It may be important to select the appropriate system depending on eye conditions such as the pattern of total and corneal higher order aberrations.

In another research, the outcomes of topography-guided and wavefront-optimized treatment were compared in patients having LASIK for myopia. In the research, patients had topography-guided LASIK in 1 eye and wavefront-optimized LASIK in the contralateral eye using the Customized Refractive Surgery Master software and Mel 80 excimer laser. Refractive (residual manifest refraction spherical equivalent (“MRSE”), HOAs, and visual (uncorrected distance visual acuity (“UDVA”)), and photopic and mesopic contrast sensitivity) outcomes were prospectively analyzed 6 months postoperatively. The study comprised 35 patients. The UDVA was 0.0 log MAR or better and the postoperative residual MRSE was ±0.50 diopter in 94.29% of eyes in the topography-guided group and 85.71% of eyes in the wavefront-optimized group (P=0.09). More eyes in the topography-guided group than in the wavefront-optimized group had a UDVA of −0.1 log MAR or better (P=0.04). Topography-guided LASIK was associated with less deterioration of mesopic contrast sensitivity at higher spatial frequencies (12 cycles per degree (“cpd”) and 18 cpd) and lower amounts of induced coma (P=0.04) and spherical aberration (P=0.04). Less stromal tissue was ablated in the topography-guided group (mean 61.57 μm±16.23 [SD]) than in the wavefront-optimized group (mean 79.71±14.81 μm) (P<0.001). Accordingly, although topography-guided LASIK and wavefront-optimized LASIK gave excellent results, topography-guided LASIK was associated with better contrast sensitivity, lower induction of HOAs, and a smaller amount of tissue ablation.

In yet another research, the mesopic contrast sensitivity (“CS”) and HOAs were compared at 3 months after femtosecond-laser in situ keratomileusis (LASIK) (FS-LASIK), wave front-guided femtosecond LASIK (WF-LASIK), and femtosecond lenticule extraction (FLEx) for the correction of myopia and myopic astigmatism. In this nonrandomized study, 332 right eyes of 332 patients were treated with FS-LASIK, WF-LASIK, or FLEx. The HOAs and mesopic CS were evaluated preoperatively and at 3 months postoperatively. At 3 months of follow-up, 98 eyes (96.1%) of the FS-LASIK group, 92 eyes (98.9%) of the WF-LASIK group, and 133 eyes (96.4%) of the FLEx group had an uncorrected distance visual acuity of 20/20 or better. The HOAs improved from 0.34 μm during preoperative examination to 0.56 μm of the end of the follow-up in the FS-LASIK group, from 0.31 to 0.41 μm in the WF-LASIK group, and from 0.32 to 0.54 μm in the FLEx group (all P<0.01). At a spatial frequency of 12 cycles per degree, a better mesopic CS was observed in the WF-LASIK group (1.47) than in the FS-LASIK (1.36) and FLEx (1.33) groups (P<0.01); a better mesopic CS with glare was also noted in the WF-LASIK group (1.37) than in the FS-LASIK (1.25) and FLEx (1.29) groups (P<0.01). Accordingly, it was concluded that the FS-LASIK, WF-LASIK, and FLEx procedures result in comparable refractive results at 3 months postoperatively. However, there is improvement in the mesopic CS and HOAs after WF-LASIK.

In yet another research, the clinical outcomes of wavefront-guided and wavefront-optimized laser were compared in LASIK. The study population included 110 eyes of 55 patients with myopia with and without astigmatism. One eye of each patient was randomized to undergo wavefront-guided LASIK by the AMO Visx CustomVue S4 IR excimer laser system; the fellow eye received wavefront-optimized LASIK by the Alcon Allegretto Wave Eye-Q 400 Hz excimer laser system. Corneal flaps were constructed using the Intralase FS 60 Hz femtosecond laser. Patients were followed at postoperative months 1, 3, 6, and 12. The study's main outcome measures were uncorrected visual acuity, stability of refractive correction, contrast sensitivity, and wavefront aberrometry. After 12 months, LASIK eyes had achieved visual acuity of 20/12.5 or better (30 eyes, 56%) in the wavefront-guided group compared to those receiving wavefront-optimized treatment (22 eyes, 41%) (P=0.016). Average spherical equivalent refractions were −0.13±0.46 diopters in wavefront-guided eyes whereas in wavefront-optimized eyes the refractions were −0.41±0.38 diopters at 12 months. Wavefront-guided eyes also achieved better best-corrected visual acuity at both the 5% and 25% contrast levels (P=0.022 and P=0.004, respectively). There were no differences in levels of residual astigmatism (P=0.798) or in higher order aberrations (P=0.869). It was concluded that both wavefront-guided and wavefront-optimized treatments were able to correct myopia safely and effectively in eyes with and without astigmatism. However, wavefront-guided treatment platforms appeared to offer significant advantages in terms of residual refractive error, uncorrected distance acuity and contrast sensitivity.

In yet another research, wavefront (WF)-guided and WF-optimized LASIK in myopes were compared. A total of 72 eyes of 36 participants with myopia with or without astigmatism were use as subjects. Participants were randomized to receive WF-guided or WF-optimized LASIK with the WaveLight Allegretto Eye-Q 400-Hz excimer laser platform. LASIK flaps were created using the 150-kHz IntraLase iFS. Evaluations included measurement of uncorrected distance visual acuity (UDVA), corrected distance visual acuity (CDVA), <5% and <25% contrast sensitivity, and WF aberrometry. Patients also completed a validated questionnaire detailing symptoms on a quantitative scale. The frequency with which the WF-guided and WF-optimized groups achieved postoperative UDVA of ≥20/16 or ≥20/20 and the frequency with which the groups lost 1 or 2 or more lines or maintained their preoperative CDVA were not statistically different from each other (all P>0.05). The frequency with which the WF-guided group attained a refractive error within ±0.25 diopters of emmetropia was higher than in the WF-optimized group (67.6%, 95% confidence interval [CI], 50.4-84.8 vs. 41.2%, 95% CI, 23.2-59.2; P=0.03). The WF-guided group's mean UDVA was better than the WF-optimized group's UDVA by approximately 1. Early Treatment Diabetic Retinopathy Study line (−0.17±0.11 logarithm of the minimum angle of resolution [log MAR], slightly <20/12 Snellen vs. −0.13±0.12, slightly >20/16; P=0.05). There were no statistically significant differences in contrast sensitivity, astigmatism, coma, or higher-order root mean square error between the groups (all P>0.05), but the WF-guided group had less trefoil compared with the WF-optimized group (0.14±0.07 vs. 0.20±0.09; P<0.01). There were no statistically significant differences in subjective parameters between the groups (all P>0.05). It was concluded that wavefront-guided and WF-optimized LASIK using the Alcon WaveLight Allegretto Eye-Q 400-Hz excimer laser platform provide similar results in myopic patients; however, the WF-guided approach may yield small gains in visual acuity, predictability, and HOAs.

In yet another research, visual performance of wavefront-guided LASIK with iris-registration (Wg-LASIK group) and conventional LASIK (LASIK group) one year after surgery was compared and the correlation between wavefront aberrations and visual performance was analyzed. Eight hundred and fifty-two myopic eyes of 430 patients were enrolled in this research and divided into two groups: Wg-LASIK group (436 eyes) and LASIK group (416 eyes). A Wavescan Wavefront aberrometer was used to analyze Zernike coefficients and the root-mean-square (RMS) of higher order aberrations, and Optec 6500 visual function instrument was used to measure contrast sensitivity (“CS”) before and 3, 6, 12 months after surgery. The mean spherical equivalent (SE) in Wg-LASIK group was significantly better than those in LASIK group one year after surgery (P=0.024). Wg-LASIK eyes showed better CS values than LASIK eyes at all spatial frequencies with and without glare after surgery (P all <0.01). Moreover, the increase of higher RMS (RMSh), coma, RMS3, RMS4, RMS5 in Wg-LASIK group were significantly lower than those in LASIK group 1 year after surgery (P all <0.05). The increase of coma, spherical aberration (SA), RMS3 and RMS4 in Wg-LASIK, and coma and RMS3 in LASIK group were negatively correlated with reduction of contrast sensitivity 1 year after surgery. It was concluded that a significant better visual performance is got in Wg-LASIK group compared with LASIK group 1 year after surgery, and the Wg-LASIK is particularly suitable for eyes with high-magnitude RMSh.

In yet another research, outcomes of customized/wavefront guided with conventional ablation in myopic patients were compared with or without astigmatism undergoing laser in situ keratomileusis. Sixty-eight eyes of 34 myopic patients with similar refractive error in both eyes were included. One eye was randomly selected to undergo conventional and the fellow eye customized ablation. Surgery was performed using the Technolas 217z laser (Bausch & Lomb, Surrey, UK). Uncorrected visual acuity, manifest refractive spherical equivalent (“MRSE”), astigmatism, aberrometry and contrast sensitivity were recorded pre and 3 months postoperatively. Mean MRSE treated in the conventional and customized groups were 3.77±1.61 diopters and −3.83±1.59 diopters respectively. Three months postoperatively there was no significant difference between the groups in mean MRSE (p=0.99) or cylinder (p=0.56). The factor increases in postoperative total higher order aberrations (HOAs) was less in the customized (1.32) compared with the conventional (1.54) treatment group but did not reach statistical significance (p=0.08). Scotopic contrast sensitivity decreased significantly postoperatively in the conventional but not in the customized treatment group. It was concluded that visual acuity and refractive error outcomes were similar in both treatment groups and no patient preference was observed. Customized ablation was associated with a smaller but not statistically significant postoperative increase in HOAs, better preservation of scotopic contrast sensitivity, quicker treatment time and removal of less corneal tissue

In another research, postoperative outcomes of a new aspheric LASIK system, which applies an index for corneal asphericity (Q-value), were compared with outcomes of the conventional LASIK procedure. Twenty-eight eyes of 15 consecutive patients (mean age, 36.4+/−5.8 years) underwent aspheric LASIK (As-LASIK group), and 33 eyes of 18 consecutive patients (mean age, 32.9+/−8.3 years) underwent conventional LASIK (Con-LASIK group). Both procedures were performed with a Moria LSK-One microkeratome and a Bausch and Lomb Technolas 217-z100 excimer laser. Preoperative mean spherical equivalent refraction values were −5.13+/−1.23 diopters (D) and −5.63+/−0.88 D in the As-LASIK and Con-LASIK groups, respectively. Higher order aberrations were measured, and contrast sensitivity was assessed at 3 months after the procedure, and these, along with safety, efficacy, and predictability, were compared between the two procedures. Conventional LASIK significantly increased higher order aberrations and reduced contrast sensitivity, whereas As-LASIK did not increase spherical-like aberrations or alter contrast sensitivity. It was concluded that apheric LASIK may be a better laser technique than Con-LASIK, with less postoperative increase in spherical-like aberrations and better control over contrast sensitivity.

LASIK has become an efficient and commonly performed procedure to reduce refractive errors. In order to further increase the postoperative visual quality, the wavefront-guided refractive surgery has been a research hotspot in customized surgery. A research was conducted to compare the visual acuity, higher-order aberration, and contrast sensitivity of wavefront-guided LASIK with iris-registration and conventional LASIK. Two hundred and eleven myopic eyes of 109 patients were enrolled in this prospective study and randomly divided into two groups: the wavefront-guided LASIK (wg LASIK) group (94 eyes) and conventional LASIK group (117 eyes). A Wavescan Wavefront aberrometer was used to analyze Zernike coefficients and the root-mean-square (RMS) of higher order aberrations with 6.0 mm pupil size, and Optec 6500 visual function instrument was used to measure contrast sensitivity (CS) under 5 spatial frequencies before and after surgery in both groups. The uncorrected visual acuity (UCVA) and the mean spherical equivalent (SE) in wg LASIK group were significantly better than those in conventional LASIK (UCVA, z=2.339, P=0.019; SE, t=2.838, P=0.005) at 3 months after surgery. Moreover, the increase in Z(3)(−3), Z(3)(1), Z(3)(3), Z(4)(0), Z(5)(−1), Z(5)(1), Z(5)(5) and Z(6)(−6) in wg LASIK group was statistically smaller than that in conventional LASIK group (P<0.05). In wg LASIK group, eyes with a higher amount of the preoperative RMS of the higher order aberrations (RMSh=0.30 microm) showed a statistically lower increase (13.5%) than those in conventional LASIK group at 3 months after surgery (33.3%) (P=0.004). And the values of 4th order spherical aberration (4thSA) and the root mean square of 6th order aberration (RMS6) in wg LASIK group were significantly lower than those in conventional group in eyes which had higher preoperative astigmatism (=1.0D) (4thSA, P=0.03; RMS6, P=0.02). Wg LASIK group showed better CS values than the correspondingly preoperative values at all spatial frequencies with and without glare at 3 months after the surgery while conventional LASIK group displayed reduced CS values except for 1.5 and 3 cycles per degree with glare. The differences between the two groups were statistically significant (P<0.001). It was concluded that Wavefront-guided LASIK with iris-registration is efficient to reduce higher order aberrations especially spherical and coma aberrations, and to improve postoperative visual acuity and contrast sensitivity compared with conventional LASIK. The application of wavefront-guided LASIK with iris-registration is particularly suitable for eyes with higher preoperative RMSh values and eyes with higher preoperative astigmatism.

In another research, outcomes after LASIK surgery using the conventional LADARVision4000 laser and the wavefront-guided LADARWave CustomCornea wavefront system were compared. A prospective study was performed involving 140 myopic eyes receiving conventional or CustomCornea LASIK between May and October 2003. The preoperative manifest spherical equivalent refraction was limited to myopia < or =−7.00 diopters (D). The preoperative manifest cylinder was limited to < or =−2.50 D of astigmatism. Patients were evaluated for 3 months following surgery. Results evaluated were uncorrected visual acuity (UCVA), best spectacle-corrected visual acuity, manifest refraction, dilated wavefront measurements, contrast sensitivity, and patient responses to subjective questionnaires. For the CustomCornea eyes at 3 months, 80% (70/87) had UCVA > or =20/20 and 95% (83/87) had UCVA > or =20/25. For the conventional eyes at 3 months, 45% (9/20) had UCVA > or =20/20 and 80% (16/20) had UCVA > or =20/25. At the 3-month postoperative visit, 85% (74/87) of the CustomCornea eyes and 55% (11/20) of the conventional eyes were within +/−0.50 D of their intended correction. At 1 and 3 months, the CustomCornea treated eyes had a statistically significant lower mean increase in higher order aberrations than conventionally treated eyes (P<0.05). It was concluded CustomCornea wavefront-guided LASIK surgery appears safe and effective and provides clinical benefits that appear to exceed those of conventional LADARVision surgery.

One aim of corneal refractive surgery is to correct defocus and astigmatism. In the process of correcting lower order aberrations (such as as myopia (nearsightedness), hyperopia (farsightedness), and astigmatism, which are correctable with glasses), higher order ocular aberrations increase. To evaluate the effectiveness of wavefront-guided laser in situ keratomileusis (LASIK) in reducing the increase of higher order aberration, aberrational change after LASIK with conventional and wavefront-guided customized ablation were compared. Our study included 48 eyes of 24 patients. We performed conventional LASIK in one eye (Group 1) and wavefront-guided customized ablation in the other eye (Group 2). Ocular aberration was measured with the Zywave, a type of Shack-Hartmann aberrometer. We then compared low and high order aberrations, contrast sensitivity, visual acuity, corneal topography, and manifest refraction preoperatively and postoperatively at 1 and 3 months. Uncorrected visual acuity improved to more than 20/20 in two eyes in the conventional ablation group and in five eyes in the customized ablation group. In the conventional ablation group, Root-mean-square for higher order (RMS(H)) was 0.215 preoperatively, 0.465 (216.3%) at 1 month, and 0.418 (194.4%) at 3 months. In the customized ablation group, RMS(H) was 0.207 preoperatively, 0.380 (183.6%) at 1 month, and 0.371 (179.2%) at 3 months after LASIK. Mesopic contrast sensitivity in the customized ablation group was higher than that in the conventional ablation group, but this change was not statistically significant. It was concluded that wavefront-guided customized ablation reduced the increase of high order aberrations resulting from LASIK. In terms of visual acuity, patient preference, and mesopic contrast sensitivity, wavefront-guided customized ablation produced slightly—but not statistically significant-better results.

In order to address such a need, this invention describes: a) devices, systems, and methods to customize an IOL by applying patient-specific eye information such as ocular and corneal aberration measurements, including but not limited to ocular-spherical equivalent, corneal astigmatism (horizontal and vertical), spherical aberration, comatic aberration (horizontal and vertical), trefoil, and quadrafoil, to the IOL; b) devices, systems, and methods that use various materials, light sources such as lasers, and other processes herein described to manufacture and ship the customized lens; and c) devices, systems, and methods to align and place the lens into a patient's eye during surgery.

In some embodiments, using various devices, systems, and methods, patient-specific eye information, such as their ocular and corneal aberrations, is applied to IOLs during their manufacturing to create customized IOLs that are “fingerprinted” to correct the patient's aberrations and thereby yield supernormal vision. Not only can the customized IOLs correct the patient's aberrations, they can also correct astigmatism in novel ways by correcting asymmetric astigmatism, skewed astigmatism, and/or irregular astigmatism. For example, by customizing the IOL for patient specific astigmatism, astigmatic correction/neutralization while allowing haptics to be placed superiorly and inferiorly (as opposed to currently available toric lenses whose haptics that need to be oriented based on patient astigmatism) may be achieved. The IOLs can use topographic, tomographic, Scheimpflug, ray tracing, Hartmann-Shack, Fourier waveform, optical path difference, or any other kind of analysis to customize the IOLs to compensate for corneal aberrations or irregularities. In some embodiments, the spherical and cylindrical correction may also be optimized within 0.05 or 0.1 or 0.2 diopter steps/increments.

Such customized IOLs may be used for cataract surgery or refractive lens exchange for any age patients, and also for intraocular contact lenses (“ICLs”) which are generally used for young patients between the age of 18 and 45, who are not candidates for LASIK or PRK, but are interested in visual acuity without glasses or contact lenses.

The process of getting a customized IOL surgically placed in the patient's eye begins at the preoperative evaluation. In some embodiments, at the preoperative visit, different eye characteristics, such as axial length, corneal coma, spherical aberration, trefoil, quadrafoil, etc., are measured using processes such as biometry, corneal topography, Scheimpflug photography, Arcscan imaging, ultrasound biomicroscopy, posterior corneal astigmatism assessment, corneal aberrometry, etc. Biometry measurements may be conducted using various methods such as Lenstar, IOL Master, Pentacam AXL, Ziemer G6, Argo Movu, etc. Corneal topography may be measured using various systems including but not limited to Arcscan, Ellex or Sonomed UBM, Alcon: Vario Topolyzer, Alcon: Verion, Bausch+Lomb: Orbscan II, Carl Zeiss Meditec: Atlas 9000, EyeQuip: Keratron corneal topographers, EyeSys Vision: Vista, System 3000, i-Optics: Cassini, Nidek: OPD-Scan III refractive power/corneal analyzer (distributed by Marco), Oculus: Pentacam (HR or AXL), Easygraph, Keratograph 4, Keratograph 5M, Medmont Instruments: E300, S4Optik: S4OPTIK MODI 02, Schwind: Sirius+Peramis, Tomey USA: TMS-4N, Topcon Medical Systems: Aladdin biometer/topographer, Tracey Technologies: iTrace aberrometer/topographer, and Ziemer Ophthalmic Systems: Galilei. Other Placido disc, Scheimpflug, and color light-emitting diode (“LED”) reflection topography measurement devices may also be used for cornea-specific higher order aberration measurements. Posterior corneal astigmatism may be measured using Pentacam, Ziemer G6, Orbscan, etc. Corneal aberrometry may be measured using Marco OPD III, iTrace, Pentacam, Ziemer G6, Schwind Sirius or Peramis, etc. Such methods can capture some combination of corneal topography, posterior corneal surface astigmatism, and cornea-specific aberrations. Generally, corneal topography measurements of cornea-specific higher order aberrations are scheduled to take place during at least 2 preoperative visits to confirm repeatability, which are scheduled at least 1 week before surgery.

In some embodiments, once the cornea-specific higher order aberrations are measured by the surgeons and the measurements are then sent to an IOL manufacturer. The preoperative, patient specific data may be sent by surgeons via HIPAA-compliant portal. Generally, a doctor, designee, or the hospital will collect all the relevant information about the patient's eye and put it onto a worksheet or manufacturer interface with patient and surgical center information. Once the manufacturer receives the measurements, they manufacture the customized IOLs according to the patient-specific measurement data. The benefits of producing an IOL that is specific to the particular eye of a particular patient include the ability to orient the astigmatic correction favorably with respect to the haptics. It is known that placement of one optic-haptic junction at an inferotemporal region, corresponding to approximately 8 o'clock for a right eye or approximately 4 o'clock for a left eye, minimizes negative dysphotopsia. Current toric lenses link the axis of the positive cylindrical correction to the haptic-optic junction, forcing surgeons to orient the haptics in a suboptimal manner in order to properly orient the axis of the toric IOL with respect to the refractive error in the patient's cornea. The IOLs as described in the present application permit independent specification of haptic placement and the orientation of the cylinder correction, allowing correction of a patient's cornea-specific astigmatism while allowing independent localization of haptics to prevent dysphotopsias. Further benefits of an IOL that is specific to the particular eye of a particular patient include astigmatic correction that can correct skewed or asymmetric corneal astigmatism that cannot be corrected using prior art toric IOLs.

Various materials such as silicone (e.g., PDMS through 3^{rd} or 4^{th} generation silicones), acrylic (e.g., methylmethacrylate), AcrySof IQ IOL BioMaterial by myalcon), Collamer by STAAR Surgical, collagen-polyHEMA, Visian ICL Products, etc. may be used to manufacture customized IOLs. Conventional IOLs of these materials (e.g., single-piece Acrysof, Tecnis, Envista, Hoya) could be used as lens blanks to manufacture customized IOLs. Other materials, such as polyurethane, latex, VHB and Ecoflex, and diverse tough hydrogels including polyacrylamide-alginate, polyacrylamide-hyaluronan, polyacrylamide-chitosan, polyethylene glycol diacrylate-alginate and polyethylene glycol diacrylate-hyaluronan, metamaterials made from titanium dioxide, thin silver film, or indium within polyacrylamide that facilitate the objectives of the invention may also be used. In some embodiments, the potential refractive index of these manufacturing materials may range between 1.338 to 1.670.

Various known methods such as cast molding, injection molding, lathing or cryolathing, and/or lithography may be used to manufacture the IOLs.

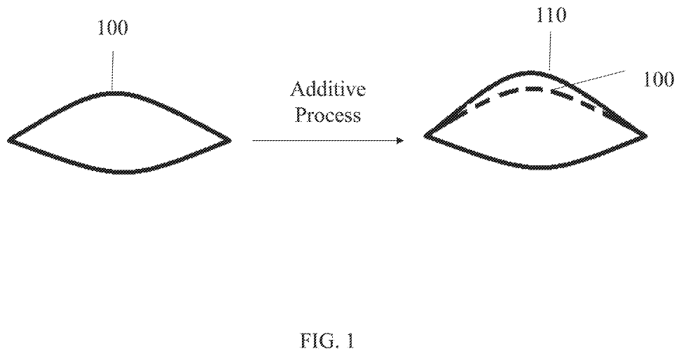

FIG. 1 illustrates an additive process for customizing IOLs according to some embodiments of the present invention. The additive process may include adding materials to the anterior surface, the posterior surface, or both surfaces of a lens blank 100. The IOLs may be additively customized by selecting the lens blank; adding material to the anterior surface, the posterior surface, or both surfaces of the lens blank to generate or move refracting surfaces to create a lens; matching the lens 110 with a patient's specifications, such as corneal topography visual axis, corneal aberrations, ocular biometry (white to white, anterior chamber depth), estimated lens position (based on UBM and/or OCT interferometry), and posterior corneal shape, with sufficient precision to correct aberrations to the desired order within the desired tolerance; and finalizing the lens 110 when it best matches the patient's specifications. Matching the lens 110 may include comparing, using generally known methods, the final power of the lens with the patient-specific measurement data to ensure accuracy. The lens blank 100 may include any of the aforementioned IOL manufacturing materials. The lens blank 100 may be selected based on the desired properties of the customized IOL. The lens blank 100 may be selected manually or using a non-transitory medium. In some embodiments, the non-transitory medium may select the lens blank 100 based on a user input that is a function of patient-specific measurement data. In some embodiments, the lens is said to best match the specifications of the particular eye of the particular patient when the lens achieves a precision of 0.05 D or 0.1 D increments in sphere and cylinder and increments of 0.05-micron root mean square error in total high-order aberrations or in any specific aberration (e.g., spherical aberration, horizontal or vertical coma, secondary astigmatism, trefoil, quadrafoil). In some embodiments, the spherical aberration offset of 0.1 to 0.3-micron root mean square error may be included in the central 2 or 3 mm zone of the customized IOL's optic to improve contrast sensitivity and/or permit extended depth of focus. In some embodiments, the additive process may be performed manually or with the help of a non-transitory medium.

In some embodiments, additive methods may include 3-D printing and/or ink jet printing of the additional material and applying a liquid on top of the lens blank's 100 surface and polymerizing that liquid so that it solidifies on the surface and creates a required additive layer on the surface of the lens blank 100. In some embodiments, the additive process may be numerically controlled. In some embodiments, when additive methods are used, particularly with a stiff lens blank material, the lens 110 may require a finishing step that makes the lens's surface optically smooth. In some embodiments, the finishing step may include surface heating by exposing the lens 110 to infrared light to raise the temperature of the lens material within a desired distance of the lens's surface, such as 50 microns. In other embodiments, the finishing step may include passing the lens 110 through a heating step, which may include autoclaving the lens 110 for sterilization. In yet another embodiment, the finishing step may include solvent vapor annealing. In yet another embodiment, the finishing step may include dipping the lens 110 into a liquid medium that has a plasticizing effect on the lens 110 material. In yet another embodiment, the finishing step may include a polishing step such as tumbling, which is frequently used in IOL manufacturing.

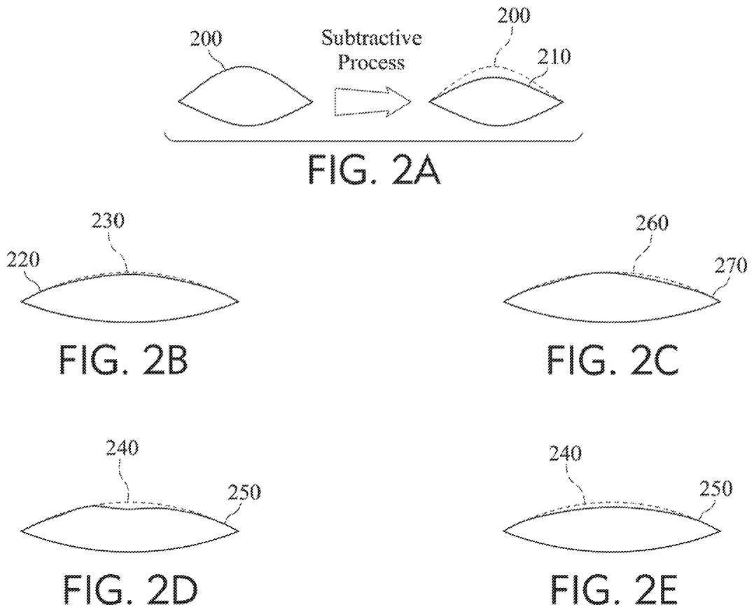

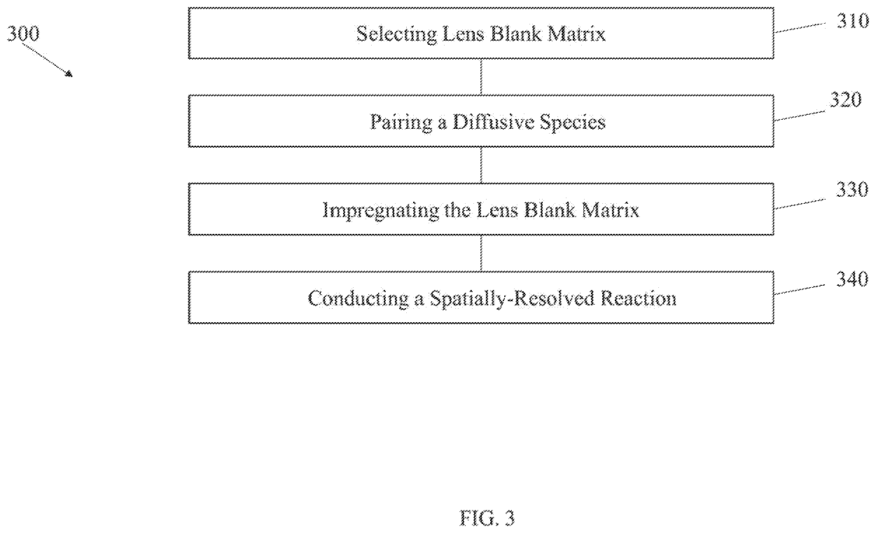

FIG. 2A illustrates a subtractive process for customizing IOLs according to some embodiments of the present invention. The subtractive process may include removing material from the anterior surface, the posterior surface, or both surfaces of a lens blank 200. The IOLs may be customized by selecting a lens blank; removing material from the anterior, posterior, or both surfaces of the lens blank 200 to generate refracting surfaces and create a lens; matching the lens 210 with a patient's specifications, such as corneal topography, visual axis, corneal aberrations, ocular biometry (white to white, anterior chamber depth), estimated lens position (based on UBM and/or OCT interferometry), and posterior corneal shape, with sufficient precision to correct aberrations to the desired order; and finalizing the lens 210 when it best matches the patient's specifications. Matching the lens 210 may include comparing, using generally known methods, the final power of the lens with the patient-specific measurement data to ensure accuracy. The lens blank 200 may include any of the aforementioned IOL manufacturing materials. The lens blank 200 may be selected based on the desired properties of the customized IOL. The lens blank 200 may be selected manually or using a non-transitory medium. In some embodiments, the non-transitory medium may select the lens blank 200 based on a user input that is a function of patient-specific measurement data. In some embodiments, the subtractive process may include machining with cutting tools, such as a small diameter end mill. In some embodiments, the subtractive process may include using fine particles accelerated to a speed such that the particles remove material from the lens blank's surface. In some embodiments, the subtractive process may include using laser ablation. In some embodiments, the subtractive process can be performed at a temperature that is below the glass transition temperature of the lens blank 100 material. In some embodiments, the subtractive process can be numerically controlled. FIG. 2B illustrates lenses 220, 230, 250, and 250 that have all undergone a subtractive process. Like the additive process described above, when a subtractive process is used to customize an IOL, particularly with a relatively stiff, yet foldable, lens blank material, the lens 210 may require a finishing step that makes the surface optically smooth. Similar finishing steps, as described for the additive process, may be used. In some embodiments, the subtractive process may be performed manually or with the help of a non-transitory medium.

FIGS. 2B-2D illustrate four lens blanks 220, 240, 260, and 280 that have undergone subtractive IOL customizing process. As illustrated, the subtractive process may be used to create lenses 230, 250, 270, and 290 with different profiles as per the patient-specific measurement data.

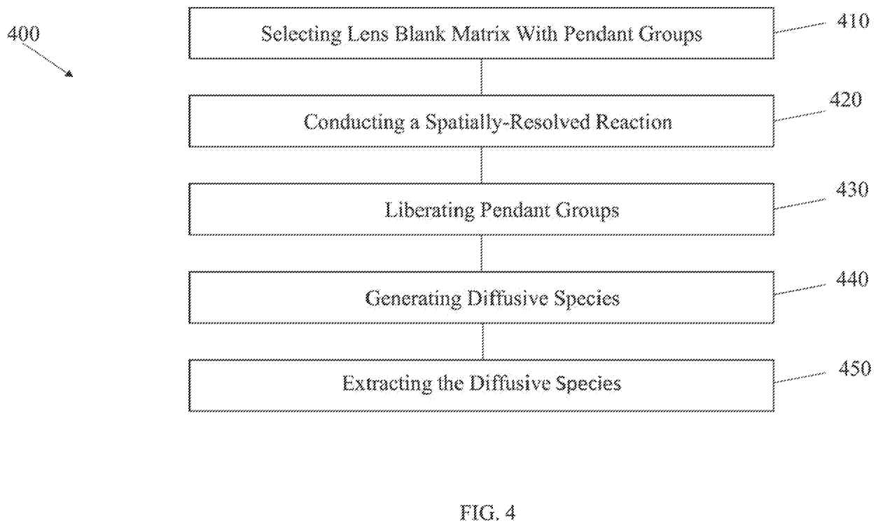

FIG. 3 illustrates a block diagram of an internal additive process 300 for customizing IOLs in a spatially-resolved manner according to some embodiments of the present invention. The internal additive process 300 may include selecting a lens blank matrix 310, pairing the lens blank matrix with a diffusive species 320, impregnating the diffusive species into the lens blank matrix 330, conducting a spatially-resolved reaction 340 using a spatially-resolved stimulus to permanently incorporate some of the diffusive species in the lens blank matrix to create a lens. The resulting shape of the lens and the spatially-resolved reaction 340 changes the refractive index of the IOL and customizes it. The lens blank matrix may include any of the aforementioned IOL manufacturing materials. The lens blank matrix may be selected based on the desired properties of the customized IOL. The lens blank matrix may be selected manually or using a non-transitory medium. In some embodiments, the non-transitory medium may select the lens blank matrix based on a user input that is a function of patient-specific measurement data. In some embodiments, impregnating diffusive species into the lens blank matrix 330 may include absorbing a polymerizable species into the lens blank matrix; stimulating the material to initiate polymerization in an appropriate spatially-resolved pattern for an appropriate amount of time to polymerize a desired amount of monomer or macromer; extracting, leaching, evaporating or diffusing the unreacted, diffusive monomers or macromers; and drying the lens. In some embodiments, polymerization may be stimulated using photopolymerization in which light of a wavelength interacts with a photosensitizer to initiate polymerization. In another embodiment, polymerization may be stimulated by thermal polymerization in which spatially-resolved temperature patterns may be created by infrared, microwave, or ultrasound irradiation. In some embodiments, polymerization may also be stimulated using the methods described in U.S. Pat. No. 6,905,641, which is incorporated by reference in its entirety. In some embodiments, polymerization of impregnated diffusive species may be numerically controlled. In some embodiments, impregnating diffusive species into the lens blank matrix 330 may include simply coating the lens blank matrix with the diffusive species. In some embodiments, after conducting the spatially-resolved reaction, the excess impregnated diffusive species is extracted to create the lens. After conducting the spatially-resolved reaction and/or extracting the excess impregnated diffusive species, the lens may be finished using similar finishing steps, as described for the additive process. In some embodiments, the internal additive process may be performed manually or with the help of a non-transitory medium.

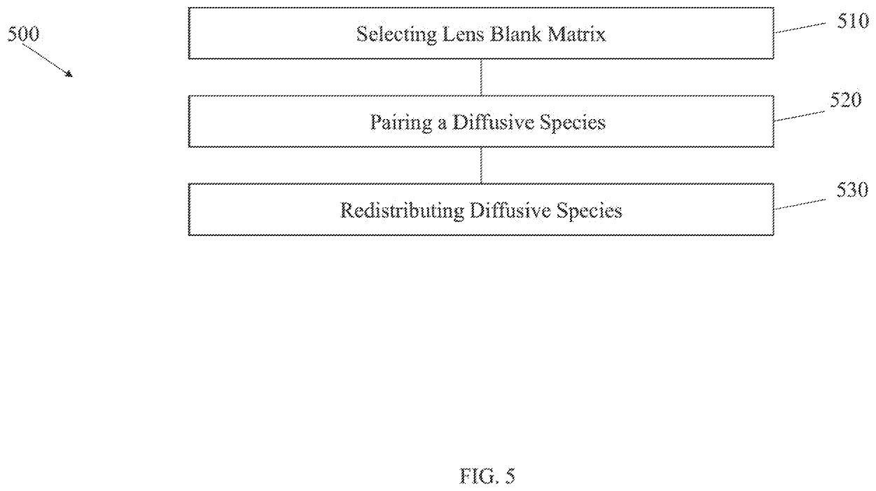

FIG. 4 illustrates a block diagram of an internal subtractive process 400 for customizing IOLs in a spatially-resolved manner according to some embodiments of the present invention. The internal subtractive process 400 may include selecting a lens blank matrix 410 that may include a network with pendant groups that can be cleaved, conducting a spatially-resolved reaction 420 using a spatially-resolved stimulus liberating some of the pendant groups 430, generating diffusive materials 440, and extracting the diffusive materials 450. The resulting shape of the lens blank and the spatially-resolved reaction 420 changes the refractive index of the IOL and customizes it. The lens blank matrix may include any of the aforementioned IOL manufacturing materials. The lens blank matrix may be selected based on the desired properties of the customized IOL. The lens blank matrix may be selected manually or using a non-transitory medium. In some embodiments, the non-transitory medium may select the lens blank matrix based on a user input that is a function of patient-specific measurement data. The spatially-resolved reaction 420 may include modifying the lens blank matrix's chemical structure by altering the chemical bonds of the lens blank matrix material, which in turn renders some of the lens blank matrix material diffusive so that it may be extracted from the lens blank matrix to create a lens. Extracting the resulting diffusive species 450 from the lens blank matrix to create a lens may be numerically controlled. In some embodiments, extracting the diffusive species 450 from the lens blank matrix may be accomplished through photoactivation. In photoactivation, a light of a wavelength that interacts with a photosensitive moiety in the material of the lens blank matrix causes a photochemically labile chemical bond to break releasing a side group that is then extracted. In another embodiment, extracting the diffusive materials 450 from the lens blank matrix may be accomplished through thermal-activation. In thermal-activation, spatially-resolved temperature patterns may be created by infrared, microwave, or ultrasound irradiation that activate degradation of thermally labile chemical bonds in the lens blank matrix material that breaks and releases a side group that is then extracted. The resolved reaction may then be completed to make the shape permanent. In some embodiments, the lens may then be finished using similar finishing steps, as described for the additive process. In some embodiments, the internal subtractive process may be performed manually or with the help of a non-transitory medium.

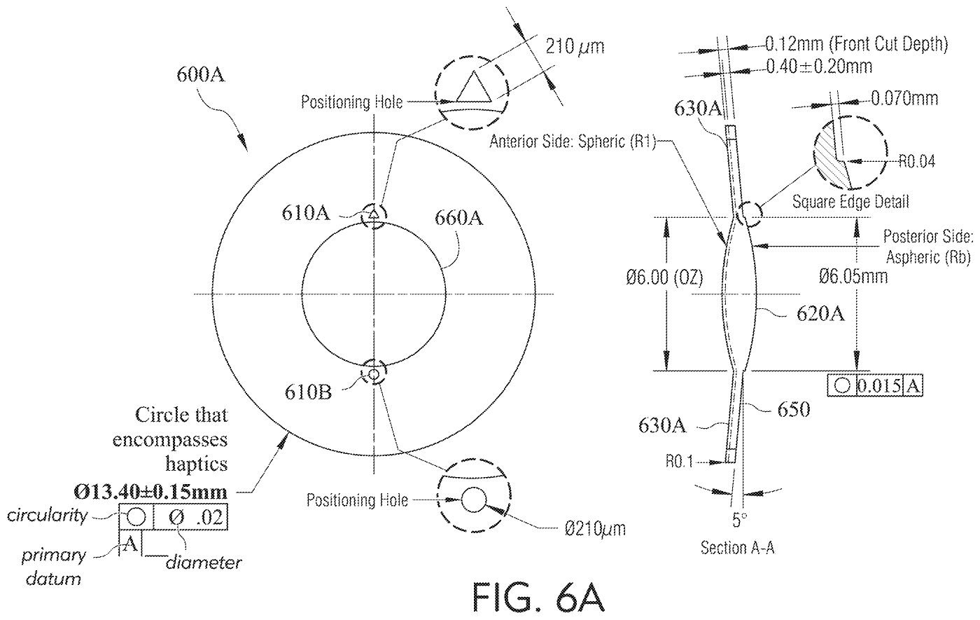

FIG. 5 illustrates a process of an internal redistributive process 500 for customizing IOLs in a spatially-resolved manner according to some embodiments of the present invention. The internal redistributive process 500 may include selecting a lens blank matrix 500, which may include a fully self-contained elastomer with diffusive species; conducting a spatially-resolved reaction 520 to permanently incorporate some of the diffusive species in the lens blank matrix material; and allowing the remaining diffusive species to redistribute 530 in response to a composition gradient that was created by the reaction and create a lens. The resulting shape of the lens and/or the spatially-resolved change of the refractive index in the IOL customizes it. The lens blank matrix may include any of the aforementioned IOL manufacturing materials. The lens blank matrix may be selected based on the desired properties of the customized IOL. The lens blank matrix may be selected manually or using a non-transitory medium. In some embodiments, the non-transitory medium may select the lens blank matrix based on a user input that is a function of patient-specific measurement data. In some embodiments, the spatially-resolved reaction 520 to permanently incorporate some of the diffusive species in the lens blank matrix material may be numerically controlled. In some embodiments, the spatially-resolved reaction may be achieved using photoactivation. In photoactivation, a light of a wavelength interacts with a photosensitive moiety in the lens blank matrix to cause polymerization or grafting of the diffusive species so that they become permanently a part of the lens material. In thermal-activation of the spatially-resolved reaction, spatially-resolved temperature patterns may be created by infrared, microwave, or ultrasound irradiation to cause polymerization or grafting of the diffusive species so that they become permanently a part of the lens material. When the desired shape of the lens and/or spatially-resolved change of the refractive index in the lens is achieved, the fully self-contained elastomer may be reacted fully to make the shape and refractive index permanent. That is, all the remaining diffusive species are polymerized to “lock in” the shape and/or spatial distribution of refractive index. After “lock in” negligible diffusive species remain in the lens. In some embodiments, “lock in” is achieved by exposing the entire lens to light of a wavelength, intensity and duration that causes photopolymerization to incorporate all of the remaining diffusive species into the matrix. In other embodiments, “lock in” is achieved by heating the entire lens to thermally polymerize all of the remaining diffusive species into the matrix. In some embodiments, the heating of the entire lens is achieved using infrared light. In other embodiments, the heating of the entire lens is achieved during autoclaving the lens for sterilization. In some embodiments, the internal redistributive process may be performed manually or with the help of a non-transitory medium.

In some embodiments, diffusive species, when separate from a lens blank matrix, are liquids at temperatures ideal for impregnating the lens blank matrix and subsequent extraction from the lens blank matrix or redistribution within the lens blank matrix, as discussed above. Diffusive species have a distribution of molar mass that may be characterized by a weight average molecular weight M_{w} and a number average molecular weight Mₙ. In some embodiments, the diffusive species have Mₙ less than 10,000 g/mol so that their diffusion is appropriately fast. For example, such a diffusive species may be impregnated into the lens blank matrix in 3 hours or less and extracted from the lens blank matrix in 3 hours or less. Diffusive species may include one or more reactive chemical moieties including but not limited to vinyl group, alkyne group, acrylate group, and methacrylate group. In some embodiments, the acrylate diffusive species is preferred. In some embodiments, the diffusive species' molecular structure is selected according to its solubility in a specific lens blank matrix. The lens blank matrix may comprise of suitable polymer mixture. For example, if the lens blank matrix is predominantly comprised of methyl-pheynl-siloxane polymer, a variety of diffusive species may be used that have a combination of dimethyl-siloxane, methyl-phenyl-siloxane, and diphenyl-siloxane provided that the composition of the diffusive species confers solubility in the chosen matrix. A skilled person would be aware that the higher the M_{w} and Mₙ of the diffusive species, the narrower the range of comonomer content that confers solubility in the matrix. In some embodiments, the solubility of the diffusive species with the lens blank matrix may be assessed by immersing a crosslinked sample of the lens blank matrix into the diffusive species. Additional examples, details, and properties of crosslinked lens blank matrix is provided in U.S. Pat. No. 7,241,009, which is incorporated by reference in its entirety.

In some embodiments, the IOLs may be customized using a method that may include selecting mold surfaces; deforming mold surfaces precisely using some force to create a shape of the anterior surface, the posterior surface, or both surfaces of the customized IOL; selecting a liquid composition; imposing the shape on the liquid composition; and causing a chemical reaction in the liquid composition to make the shape permanent prior to removal from the mold. In some embodiments, the force and energy used to deform mold surfaces may include kinetic, compression, gravitational, tension, spring, and/or electric. In some embodiments, the mold surfaces may have the capability to generate plano-convex or biconvex shapes. Various mold surfaces that are generally available may be used to create a shape for the customized IOL's surface. For example, disposable molds, as discussed in U.S. Pat. No. 5,141,678, which is incorporated by reference in its entirety, may be used. Reconfigurable mold that has a deformable surface that is deformed by an array of actuators to define a specified surface contour that will result in a desired wavefront, as discussed in U.S. Patent Publ. No. 2005/0264756, which is incorporated by reference in its entirety, may also be used. The IOLs may be customized by cast molding intraocular lenses produced from two or more dissimilar materials using disposable plastic molds as discussed in U.S. Pat. No. 6,391,230, which is incorporated by reference in its entirety. The IOLs may also be customized using injection molding as described in U.S. Pat. No. 8,663,510, which is incorporated by reference in its entirety.

In some embodiments, the IOLs may be customized using an external stimulus such as light as described in U.S. Patent Publ. No. 2003/0128336 and U.S. Patent Publ. No. 2007/0055369, which are incorporated by reference in their entireties.

In some embodiments, the manufacturer may make multiple customized IOLs, such as 5 or more IOLs, for a given patient-specific measurement data set. The manufacturer may then choose the best customized IOL that is predicted to minimize the aberration and optimize the visual acuity and contrast through an image simulator.

After the manufacturing is complete, the resulting customized IOLs, in some embodiments, may be flexible to be inserted through 3.2, 2.8, 2.4, 2.2, or smaller incisions in the eye. The customized IOLs may have an optic diameter that ranges from 5.0 to 6.5 mm. In some embodiments, an optic diameter of 6.0 mm may be preferred. The long axis of the customized IOLs may be between 11.5 to 13.5 mm. The customized IOLs' haptics may be a single piece made out of the same material as the IOL itself. In other embodiments, the haptic and the customized IOL may be made from different materials. The customized IOLs and their haptics may have different shapes and designs. For example, the haptics may be made from flexible polypropylene, polyimide, or PMMA, and/or part of a 3-piece customized IOL.

In some embodiments, after manufacturing, customizing, and labelling the IOLs, the customized IOLs are delivered to the surgical facility before the surgery. In some embodiments, the manufacturers may have a production facility to manufacture customized, patient-specific IOLs and a distribution line with the capability to deliver the customized IOLs throughout a country or internationally. In other embodiments, the manufacturers may have a production facility to manufacture customized IOLs and a distribution line with the capability to deliver the customized IOLs to a geographic region that is accessible within a particular delivery time such as 24 hours. In other embodiments, the manufacturers may have a production facility to manufacture customized IOLs and a distribution line with the capability to deliver the customized IOLs to a particular hospital network or a particular hospital on the same day. In other embodiments, the manufacturers may have a production facility to manufacture customized IOLs and a distribution line dedicated to a specific hospital or a doctor. In other embodiments, the manufacturers may have a production facility and a distribution line near a site where eye care such as consultation and/or eye surgery is performed. In other embodiments, the manufacturers may not have any distribution line but may use third party services to deliver the customized IOLs to the doctor, hospital, and/or the hospital network.

Upon receiving the customized IOLs, a doctor, designee, and/or hospital may perform quality control by crosschecking the delivered IOLs' parameters against the ordered patient-specific measurement data. Since it is very important that the correct customized IOLs are matched to the correct eye, patient, doctor, and surgical facility, Radio-Frequency IDentification (“RFID”), UPC or QWERTY code, and/or Quick Response Code (“QR”) may be matched with the patient's wrist label information to confirm the delivery. Additionally, the customized IOL boxes may also come with eye and patient information, which may be further confirmed by the doctor, designee, surgical center, or hospital. In some embodiments, an additional level of confirmation can also be achieved by the doctor, designee, surgical center or hospital by crosschecking the customized IOL's aberrometry correction profile with patient's preoperative aberrations, surgical plan, and astigmatism data.

In some embodiments, after the surgical facility and the doctor receive and verify the customized IOLs, the eye surgery may be commenced. Various tools such as Alcon Verion may be used to assess preoperative pupillary registration and improve alignment of the IOL. Other tools that facilitate the same purpose may also be used. Preoperative pupil registration assessment may include determining where the pupil is located relative to the visual axis, and/or the corneal center. The pupil's location relative to the visual axis may be defined as the line of sight from the fovea, which may be identified by the first Purkinje image. The pupil's location relative to the corneal center may be defined relative to the corneal limbus.

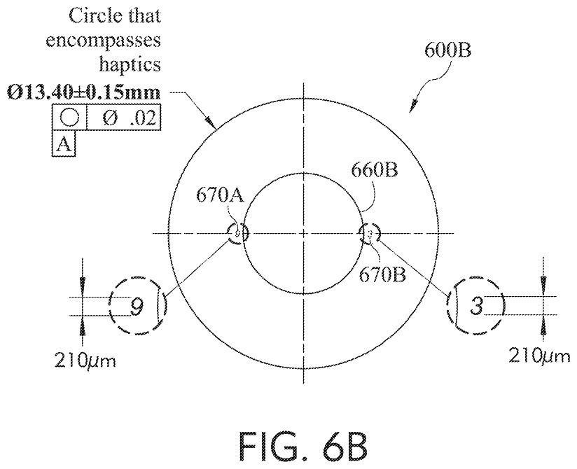

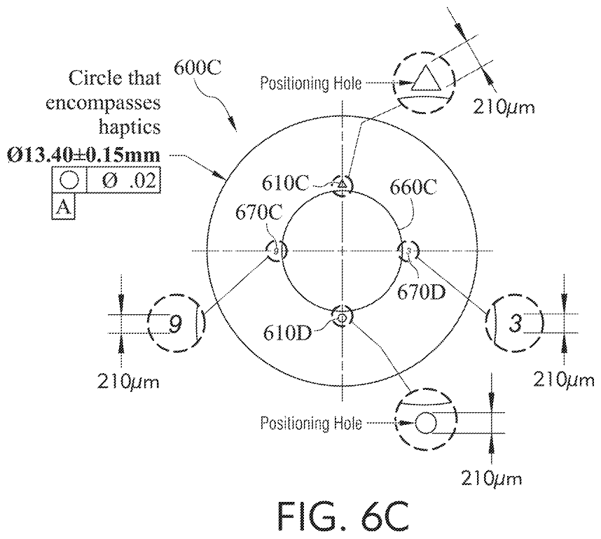

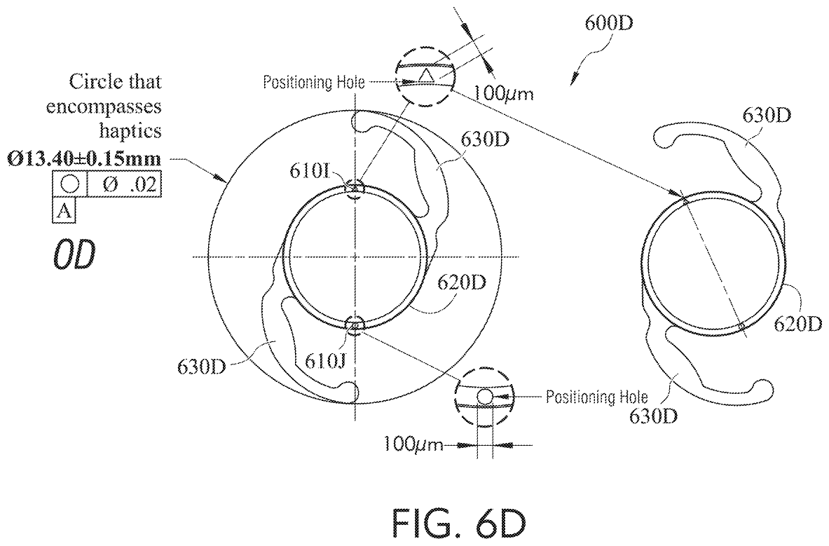

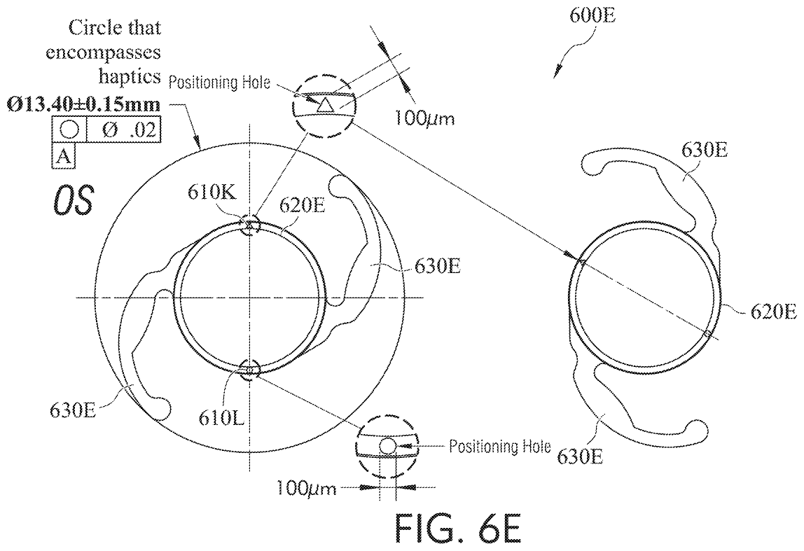

FIGS. 6A-6E illustrate exemplary customized, preoperative, patient-specific markings on customized IOLs 600A-600E. FIG. 6A illustrates a perspective and side view of a customized IOL 600A with two positioning holes 610A and 610B, along an optic border 650, to be used intraoperatively to properly orient the customized IOL. To illustrate the concept in FIG. 6A, a triangular hole is used to indicate the point along the optic border that should be oriented at the 12 o'clock direction and a circular hole is used to indicate the point along the optic border that should be oriented at 6 o'clock direction. The customized IOL 600A comprises of the optic section 620A and the haptic section 630A. In some embodiments, optic section 620A may include a spherical correction and an astigmatic correction having magnitude and orientation based on preoperative measurements of a specific eye of a specific patient. In contrast to prior art toric lenses that lack patient specific markings, in some embodiments, the lenses provide one or more features on the customized IOL for intraoperative guidance to enable the surgeon to properly orient the patient-specific IOL such that the cylinder axis of the astigmatic correction is properly oriented for the specific eye of the specific patient. In contrast to prior art phakic-IOLs, customized IOLs are the first phakic-IOLs to offer astigmatic correction. In some embodiments, the optic section 620A may include a correction of at least one high-order aberration based on preoperative measurements for the specific eye of the specific patient.

The optic section 620A may include a square edge 640 on its posterior outer edge to minimize posterior capsule opacification. The radius of the square edge 640 may be 0.04 mm. The square edge 640 may be offset from the posterior outer edge by 0.070 mm. In some embodiments, the haptic section 630A may include square posterior haptic edges. The haptics 630A may be offset from the central vertical optical axis 650 by 5 degrees. FIG. 6B illustrates a perspective view of the customized IOL 600B with positioning markings 670A and 670B, along the optic border 660C, at 3 o'clock and 9 o'clock directions, respectively. FIG. 6C illustrates a perspective view customized IOL 600C with positioning holes 610C and 610D at 12 o'clock and 6 o'clock direction, respectively, and positioning markings 670C and 670D at 3 o'clock and 9 o'clock directions, respectively. In some embodiments, the positioning markings 670A-670D may be desirably oriented with respect to the cylinder axis of the astigmatic correction. In some embodiments, the positioning markings 670C and 670D at 3 o'clock and 9 o'clock directions have dimensions along the 12 o'clock to 6 o'clock direction that is between 1.25 to 3 times its dimensions in the 3 o'clock to 9 o'clock direction.

A doctor, assistant, or nurse may apply the positioning markings 670A-670D on the eye, such as markings on 3 o'clock and 9 o'clock positions while the patient is in the sitting position. In some embodiments, the doctor, surgical technician or surgeon's designee, or nurse may also apply the positioning markings 670A-670D compensating for supine cyclotorsion, which refers to spontaneous rotation of the eye approximately 5-10° upon laying down. In some embodiments, such positioning markings 670A-670D may also be made on an image resembling the patient's eye and/or interior of the eye so that the doctors have a reference image to superimpose on the view of the patient's eye through a surgical microscope while performing the surgery. For example, such markings may be displayed to and viewed by the doctor as part of a heads-up display on operating microscopes, which can superimpose the preoperative image with such markings to guide alignment during surgery. Such positioning markings 670A-670D may be made using various devices including but not limited to a full spectrum laser. In some embodiments, the positioning markings 660A-660D may have a circumference ranging between 50 microns and 200 microns. In some embodiments, the optic section 620A may include an astigmatic correction specific in magnitude and orientation based on preoperative measurements for a specific eye of a specific patient.

FIGS. 6D and 6E illustrate a top view of the customized IOLS 600D and 600E, with optic sections 620D and 620E and haptic sections 630D and 630E, respectively. The customized IOL 600D may include positioning holes 610E and 610F. The customized IOL 600E may include positioning holes 610G and 610H. The positioning holes 610A-610D may have different geometrical shapes such as a triangle, circle, etc. Each positioning hole in a pair may have a shape that is similar or different to the shape of the other positioning hole in the pair. For example, in some embodiments, a pair of positioning holes 610A and 610B may be triangularly and circularly shaped, respectively. The triangular shape may point towards the top of the patient's head indicating an upward direction and the circular shape may be in the opposite, downward direction towards the patient's feet. The positioning holes 610A-610D may have an equivalent diameter of 190 to 210 microns. The positioning holes in 600D and 600E may have an equivalent diameter of 100 microns. The shape of the cross-section of a positioning hole is a two-dimensional, non-intersecting, convex shape. The equivalent diameter of the positioning hole is the average of the length of the minimum bisector and the length of the maximum bisector of the cross-section of the positioning hole. A bisector is a straight line that divides the area of the two-dimensional, non-intersecting, convex shape into two equal areas. The length of the bisector is the distance between the two points where the straight line intersects the perimeter of the two-dimensional, non-intersecting, convex shape. In some embodiments, the haptic sections 630A, 630D, and 630E may be positioned to minimize negative dysphotopsia when the astigmatic axis of the intraocular lens is properly oriented in the specific eye of the specific patient, such as one optic-haptic junction at approximately 8 o'clock for a right eye or at approximately 4 o'clock for left eye. In some embodiments, the positioning holes and/or the positioning markings may be made manually. In some embodiments, the positioning holes and/or the positioning markings may be made automatically using a laser cutting device. In some embodiments, the laser cutting device uses a CO2 laser. In some embodiments, the positioning holes and/or the positioning markings may be made automatically using a non-transitory medium.

During a surgery to implant a customized IOL in a particular eye of a particular patient, a corneal incision or a sclera tunnel is made. In some embodiments, the corneal incision or the sclera tunnel may be made in a temporal position. The temporal position is preferred for the corneal incision or the sclera tunnel due to advantages of minimizing surgery-induced aberrations and providing surgical-aberrations that are both small and predictable. In some embodiments, the corneal incision or the sclera tunnel is less than 3 mm long. In preferred embodiments, the corneal incision or the sclera tunnel is less than 2.5 mm long. A length of the corneal incision or the sclera tunnel that is less than 2.5 mm is preferred due to advantages of minimizing surgery-induced aberrations and providing surgical-aberrations that are both small and predictable.

If the surgery is a cataract surgery or a refractive lens exchange, a capsulotomy with a radius that is approximately 0.5 mm smaller than the radius of the optic of the customized IOL and centered on the visual axis is made. In some embodiments, the capsulotomy may be performed using one or more of the following: a femtosecond laser, capsulorhexis guide such as Verus, or a dedicated capsulotomy maker, such as Capsulaser by Excellens or Zepto from Mynosys. For example, a surgeon would prepare a capsulorhexis that has a diameter between 4.75 mm and 5.25 mm for implantation of a customized IOL having a 6 mm diameter optic. Subsequently, the customized IOL is implanted in the patient's eye using an uncomplicated cataract surgery through the temporal corneal incision or sclera tunnel. The surgeon implants, centers, and aligns the customized IOL in the capsular bag. If the surgery is a phakic-IOL implantation, the customized IOL may be placed either in front of or behind the iris of the particular eye of the particular patient.

After the customized IOL is inserted into the particular eye of the particular patient, the surgeon positions the customized IOL in the center of the pupil, either in front of or behind the iris of the particular eye of the particular patient. In some embodiments, during the surgery, the customized IOL is centered on the visual axis of the particular eye of the particular patient. In some embodiments, the customized IOL is centered manually. In some embodiments, the customized IOL is centered using one or more of the following intraoperative devices: an operating microscope to sight the using coaxial illumination, a keratoscope with flashing light, or an eyetracker such as Zeiss Callisto or Leica Proveo, or an operating microscope fitted with a Mastel intraoperative keratoscope.

In some embodiments, the IOL is implanted by the surgeon after removal of the natural lens in cataract surgery or clear lens extraction by phacoemulsification or other lens removal process. Then, the surgeon implants, centers, and aligns the IOL in the capsular bag. Then, the viscoelastic material that is present behind the customized IOL may be removed so that lens is adherent to anterior capsular bag rim and posterior capsule of the eye. The customized IOL is secured in place through the use of haptics which engage the walls of the capsular bag. The haptics can be of any conventional design. Then, the viscoelastic material that is present behind the customized IOL may be removed so that lens is adherent to anterior capsular rim and posterior capsule of the eye. In some embodiments, once the customized IOL is in place, the capsular bag is filled with a composition. When the composition forms a physical or covalent gel, it provides an anchor for the customized IOL to the capsular bag and helps in the accommodation process. More details on the composition that can be used to fill the capsular bag is provided in U.S. Patent Publ. No. 2005/0246018, which is incorporated by reference in its entirety.

In some embodiments, the customized IOL is aligned to the horizontal meridian, which is marked preoperatively or during surgery using microscope alignment systems. For example, pupillary center, and 0-degree & 180-degree meridians are marked prior to surgery on the pupillary center. The visual axis is precisely registered intraoperatively using a surgical microscope such as the Mastel keratoscope flashing light, Zeiss Callisto, Leica Proveo, or Truvision systems. During surgery, the customized IOLs may be aligned based on a registration system connection between preoperative biometry registration of eye landmarks (e.g., sclera, limbus, iris, astigmatic axis, etc.) and intraoperative microscope with heads up of 3-D visualization systems used by the surgeon. Other intraoperative instruments to assist proper lens positioning may include intraoperative aberrometry tools that illustrate optimal placement to minimize aberrations, such as Alcon ORA, Clarity Holos, etc., intraoperative microscope-integrated optical coherence tomography (“OCT”) tools that confirm optical axial location and minimize tilt such as Zeiss Rescan and Leica Enfocus, Leica Envisu, etc., and intraoperative heads-up in-ocular display systems tools, which optimize lens rotational alignment such as Zeiss Callisto, Leica DI-C800, etc. Tools, such as the Visiometrics HD Analyzer may also be used to capture preoperative angles to assist alignment of the customize IOL within the eye.

If the surgery is a cataract surgery or a refractive lens exchange, the customized IOL is secured in place through the use of haptics which engage the walls of the capsular bag. The haptics can be of any conventional design. If the surgery is a cataract surgery or a refractive lens exchange, the viscoelastic material that is present in the capsular bag behind the customized IOL may be removed. In some embodiments, the customized IOL is adherent to the posterior capsule of the eye. In some embodiments, the customized IOL is adherent to the anterior rim of the capsular bag. In some embodiments, once the customized IOL is in place, the capsular bag is filled with a composition. When the composition forms a physical or covalent gel, it provides an anchor for the customized IOL to the capsular bag and helps in the accommodation process. More details on the composition that can be used to fill the capsular bag is provided in U.S. Patent Publ. No. 2005/0246018, which is incorporated by reference in its entirety.

In some embodiments, the customized IOL may include features that indicate how to place it within the eye, such as marks on the customized IOL's optic-haptic junction to facilitate aligning it with the appropriate astigmatic axis and/or marks that indicate sinus side, temple side, and top and bottom side of the IOLs. In some embodiments, the customized IOLs may include features that prevent them from shifting or rotating after being implanted into the eye. For example, the customized IOL may have a single-piece design with a floppy haptic that reduces rotation. In other embodiments, capsule tension rings, such as from Morcher, FCI Ophthalmics, Geuder, or StabilEyes, may be used to safely ensure stability and promote centration of the customized IOL. In some embodiments, adjustable IOLs may be used that can be adjusted or rotated post-operatively using manual methods such as controlled pulses of laser radiation or micromotors to achieve improved focus and astigmatic correction as discussed in U.S. Pat. No. 5,728,155, which is incorporated by reference in its entirety.

In some embodiments, as part of internal or the manufacturer's quality control process surgeons, designee, and/or hospital may be required to document intraoperative readings at the time of implantation (prior to incision) and be required to transmit the readings to the customized IOL manufacturing company. The failure to do so may result in the IOL company disqualifying the surgeon and/or the surgical facility from using the customized IOL for a specified time period. In some embodiments, certain methods to detect post-operative refractive error and recognize patterns of post-operative refractive errors may also be provided. For example, the doctor may residual refractive aberrations to the desired order of the customized IOL in a patient in a follow-up meeting. For example, in a follow up meeting held at least once more than 30 days after the surgery and less than 365 days after the surgery, the doctor can record the uncorrected visual acuity, both for distance and near, of the patient's eye with the customized IOL. Such recorded data may be transmitted to the customized IOL manufacturing company. Failure to do so may result in that doctor not receiving the customized IOLs for a specific period as determined by the hospital or the manufacturer.

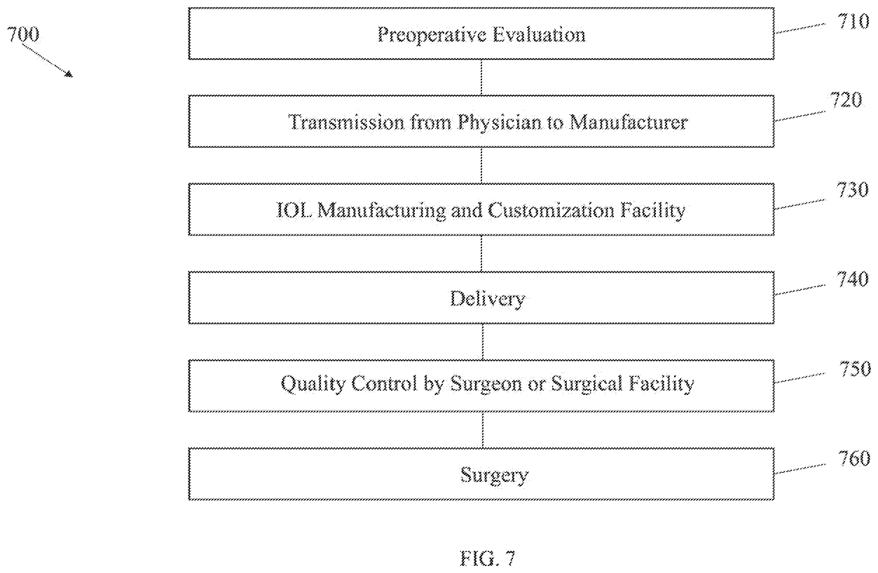

FIG. 7 illustrates a block diagram of the complete process 700 involved in a patient surgically receiving a customized IOL for a particular eye according to some embodiments of the present invention. Each of the steps may be performed according to the aforementioned details. The process 700 may include a preoperative evaluation 710 of the particular eye of the particular patient and accumulation of the patient-specific measurement data by a physician or hospital. Preoperative evaluation 710 may include axial length measurement, keratometry, anterior and posterior corneal topography, ocular biometry (white to white, anterior chamber depth), estimated lens position (based on ultrasound biomicroscopy and/or OCT interferometry), and corneal aberrations, visual axis positioning, horizontal meridian registration (using iris or sclera markers), etc. In some embodiments, the refractive errors to be corrected and the tolerance specifications may be included in the data set. For example, the patient may decide to correct defocus and astigmatism, but not any higher order aberrations. Alternatively, the patient may choose to correct defocus, astigmatism, trefoil, and tetrafoil. For each aberration to be corrected, the tolerance may be specified. Next, the measurement data set for the particular eye of the particular patient may be transmitted from the physician, designee, the operating suite or operating microscope heads-up display or linked monitor/screen, and/or hospital to the customized IOL manufacturer 720 according to any of the available transmission methods including but not limited to secure/encrypted & HIPAA-compliant patient xportal, e-mails, posts, phone calls, and couriers. The data that is transmitted from the physician, designee, and/or the hospital may be per individual or in bulk. The data that is transmitted may be sent immediately or in set time intervals. For example, the patient-specific measurement data may be sent immediately by entering it into a system that is accessible by the manufacturer or the patient-specific measurement data may be sent once a week in bulk from the physician, designee, and/or the hospital to the manufacturer using courier. The means and mode of data transmission can be any other means and mode of data transmission, as well. The physician, designee, and/or the hospital may decide what particular patient-specific measurement data to send to the manufacturer. In some embodiments, the patient-specific measurement data that is sent to the manufacturer may include at least one means to track the delivery. Next, the IOL manufacturer manufactures and customizes the IOL 730 as per any of the embodiments described above. Next, the manufacturer arranges the customized IOL to be delivered 740 to the surgeon, designee, and/or hospital using any available delivery or transmitting methods. In some embodiments, the delivery 740 may also be tracked. Any global, regional, and/or local methodologies available for package delivery may be used for transmitting patient specific data to an entity that produces the conjugate lens and delivering that specific lens to the correct hospital/doctor/surgical suite at the right time. For example, and not limitation, any of the methods described in U.S. Pat. Nos. 6,275,745 and 8,898,083, and U.S. Patent Publ. Nos. 2015/0046361, 2016/0232585, 2017/0082728, all of which are incorporated by reference herein in their entirety, may be used for transmitting patient specific data and/or delivering the customized IOL. Upon receiving the customized IOL, the surgeon, designee, and/or hospital may perform quality control 750 to ensure that the customized IOL meets the patient-specific measurement data, quality, and other requirements. The methods of performing quality control 750 may be determined by the surgeon, designee, and/or the hospital. In some embodiments, the surgeon, designee, and/or the hospital may be required to input the results of the quality control 750. In some embodiments, failure to do so may result in some penalty. Finally, as described above, the surgeon may perform the surgery 760. In some embodiments, each of the aforementioned steps may be performed manually. In some embodiments, some of the aforementioned steps may be performed manually and the rest using a non-transitory processing medium. In some embodiments, all of the aforementioned steps may be performed using a non-transitory processing medium.

In the above detailed description and in the figures, like elements are identified with like reference numerals. The use of “e.g.,” “etc.,” and “or” indicates non-exclusive alternatives without limitation, unless otherwise noted. The use of “including” or “includes” means “including, but not limited to,” or “includes, but not limited to,” unless otherwise noted.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B”, when used in conjunction with open-ended language such as “comprising” can refer, in one embodiment, to A only (optionally including entities other than B); in another embodiment, to B only (optionally including entities other than A); or, in yet another embodiment, to both A and B (optionally including other entities). These entities may refer to elements, actions, structures, steps, operations, values, and the like.

1. A customized IOL for a specific eye of a specific patient comprising:
a clear optic comprising an astigmatic axis, and
an optic border surrounding a clear lens including two positioning holes located on opposite sides of the clear optic and desirably oriented with respect to the cylinder axis of the astigmatic correction.

2. The customized IOL of claim 1, where in the holes in the optic border surrounding the clear lens are distinguishly shaped.

3. The customized IOL of claim 1, wherein the clear optic comprises a correction of at least one high-order aberration based on preoperative measurements for the specific eye of the specific patient.

4. The customized IOL of claim 3 further comprising haptics positioned based on the preoperative measurements for the specific eye of the specific patient.

5. The customized IOL of claim 4, wherein the haptics are positioned to minimize negative dysphotopsia when the astigmatic axis of the IOL is properly oriented in the specific eye of the specific patient.

6. The customized IOL of claim 3 further comprising a power with a precision of 0.05 D or 0.1 D increments in sphere and cylinder and increments of 0.05-micron root mean square error in total high-order aberrations or in any specific aberration.

7. The customized IOL of claim 3 further comprising a spherical aberration offset of 0.1 to 0.3-micron root mean square error in a central 2 or 3 mm zone of the customized IOL's optic.

8. The customized IOL of claim 1, wherein the clear optic further comprises an astigmatic correction specific in magnitude and orientation based on preoperative measurements for a specific eye of a specific patient.

9. The customized IOL of claim 2, wherein the optic border further comprises at least two distinguishable markings that have a dimension between 50 microns and 200 microns at specific azimuthal positions relative to preoperative measurements for the specific eye of the specific patient.

10. The customized IOL of claim 9, wherein the distinguishable markings are placed at the 3 o'clock and 9 o'clock positions.

11. The customized IOL of claim 1, wherein the clear optic comprises a square edge on its posterior outer edge to minimize posterior capsule opacification.

12. The customized IOL of claim 1 further comprises a haptic with square posterior haptic edges.

13. An internal subtractive method for customizing IOLs comprising:
pairing a diffusive species with a lens blank matrix with a specific desired property;
impregnating the diffusive species into the lens blank matrix;
conducting a spatially-resolved reaction between the diffusive species and the lens blank matrix; and
extracting excess diffusive species to create a lens that is customized based on preoperative measurements of a specific eye of a specific patient.

14. A method of surgically implanting a customized IOL into a patient's eye comprising:
evaluating patient-specific measurement data;
compiling patient-specific measurement data;
manufacturing the customized IOL based on patient-specific measurement data;
delivering the customized IOL to a surgeon;
matching the customized IOL to the patient-specific measurement data; and
performing an IOL insertion surgery with patient-specific alignment.

15. The method of claim 14, wherein manufacturing the customized IOL further comprises applying a subtractive process to a lens blank.

16. The method of claim 14, wherein manufacturing the customized IOL further comprises applying an additive process to a lens blank.

17. The method of claim 14, wherein manufacturing the customized IOL further comprises applying an internal additive process on a lens blank matrix.

18. The method of claim 14, wherein manufacturing the customized IOL further comprises applying an internal subtractive process on a lens blank matrix.

19. The method of claim 14, wherein manufacturing the customized IOL further comprises applying an internal redistributive process on a lens blank matrix.

20. The method of claim 14, wherein delivering the customized IOL to a surgeon further comprises utilizing any global, regional, or local methodologies available for package delivery with at least one of encryption, data security measures, or HIPAA compliance.
